(19) United States
(12) Patent Application Publication
(10) Pub. No.: US 2024/0208921 A1
(43) Pub. Date: Jun. 27, 2024

(54) Synthesis of Thiophene Derivatized Polyphenolic Calixarenes

(71) Applicant: University of Cincinnati, Cincinnati, OH (US)

(72) Inventors: Harshita Kumari, Mason, OH (US); Ajaz Ahmad Dar, Cincinnati, OH (US)

(73) Assignee: University of Cincinnati, Cincinnati, OH (US)

(21) Appl. No.: 18/540,458

(22) Filed: Dec. 14, 2023

Related U.S. Application Data
(60) Provisional application No. 63/432,491, filed on Dec. 14, 2022.

(51) Int. Cl. C07D 333/16; A01N 59/16; A61K 47/54; C09D 4/00; C09D 5/16

(52) U.S. Cl. CPC C07D 333/16; A01N 59/16; A61K 47/545; C09D 4/00; C09D 5/1625

(57) ABSTRACT

A novel composition is provided. The composition is a tetrameric reaction product of heterocyclic 3-thiophenecarboxaldehyde and an aromatic compound, the reaction product being a calix[4]arene. In one embodiment, the present invention is a thiophene functionalized calixarene selected from the group consisting of C-thiopheneresorcin[4]arene and C-thiophenepyrogallol[4]arene. In another embodiment, the thiophene functionalized calixarene further comprises silver nanoparticles.

### CROSS REFERENCE TO RELATED APPLICATIONS

This application claims priority to U.S. Provisional Application Ser. No. 63/432,491, filed Dec. 14, 2022, which application is hereby incorporated by reference in its entirety.

### TECHNICAL FIELD

The present invention relates to novel calix [4]arenes.

### BACKGROUND OF THE INVENTION

Calix[4]arenes are distinct cyclic tetrameric compounds having an alternating structure derived from the reaction of a phenolic compound with an aldehyde. The core structure is typically bowl-shaped tetrameric calix[4]arene formed from aromatic compounds having hydroxy groups and aromatic aldehydes preferably also having heteroatom present. Calix[4]arenes exist in four different conformers: cis-cis-cis, cis-cis-trans, cis-trans-cis and trans-trans-trans. Typically, the thermodynamically most favored conformer is the cis-cis-cis in which all four arene rings are flipped in the same direction such that the calix [4]arene molecule possesses a C₄ symmetry axis, in contrast to the trans-trans-trans isomer which possesses a C₂ symmetry axis.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention.

In an embodiment of the invention, the present invention is a composition having Formula 1:

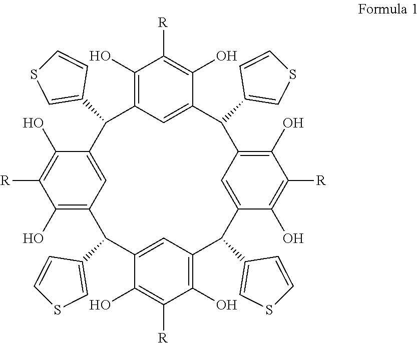

where R is H or an OH group. In another embodiment, the present invention is a tetrameric reaction product of heterocyclic 3-thiophenecarboxaldehyde and an aromatic compound, the reaction product being a calix [4]arene. In one embodiment, the present invention is a thiophene functionalized calixarene selected from the group consisting of C-thiopheneresorcin[4]arene and C-thiophenepyrogallol[4]arene. In another embodiment, the present invention is a composition having a C-thiopheneresorcin[4]arene. In another embodiment, the present invention is a composition having a C-thiophenepyrogallol[4]arene. In one embodiment, the thiophene functionalized calixarene further comprises silver nanoparticles.

In another embodiment, the present invention is a thiophene-based pyrogallol[4]arene-capped silver nanoparticles having the formula Pg₄THP₄—AgNP. In one embodiment, the present invention is a pharmaceutical drug including a composition of Formula 1. In another embodiment, the present invention is an imaging agent including a composition of Formula 1. In one embodiment, the present invention is an antibiotic coating including a thiophene-based pyrogallol[4]arene-capped silver nanoparticles having the formula Pg₄THP₄—AgNP.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 is the chemical structure of a novel calix [4]arene according to the present invention.

FIG. 2 is a schematic representation of a one-pot synthesis procedure of the present invention to produce calix[4]arenes using an acidic catalyst.

FIG. 3A is an image showing the chemical structure of C-thiophenepyrogallol[4]arene.

FIG. 3B is an image showing the chemical structure of C-thiophenepyrogallol[4 ]arene with the solvent molecules and hydrogens omitted for clarity.

FIG. 4 is a graph showing UV-vis spectra of Pg₄THP₄ (black line) and Pg₄THP₄—AgNPs (gray line) showing characteristic peaks of SPR band at 440 nm.

FIG. 5 is a graph showing the powder XRD pattern of Pg₄THP₄—AgNPs (black line) and Pg₄THP₄ (gray line) showing the presence of Pg₄THP₄ on Pg₄THP₄—AgNPs.

FIG. 6 is a series of TEM images of the Pg₄THP₄—AgNPs, synthesized with the aid of white light.

### DETAILED DESCRIPTION OF THE INVENTION

The details of one or more embodiments of the disclosed subject matter are set forth in this document. Modifications to embodiments described in this document, and other embodiments, will be evident to those of ordinary skill in the art after a study of the information provided herein.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The compositions and methods of the present disclosure can comprise, consist of, or consist essentially of the essential elements and limitations of the disclosure described herein, as well as any additional or optional ingredients, components, or limitations described herein or otherwise useful.

All percentages, parts and ratios herein are based upon the total weight of the compositions of the present disclosure, unless otherwise indicated.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “imaging agent” means a compound suitable for performing in vivo optical imaging of a region of interest in a complete (ie, intact) mammalian body.

As used herein, the term “pharmaceutical drug” means any substance which shows a medicinal effect in a patient, e.g. for curing, alleviating, or modifying clinical symptoms or causes.

As used herein, “silver nanoparticle” means a pure crystalline silver particle having a size ranging from 1 nm to 100 nm.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

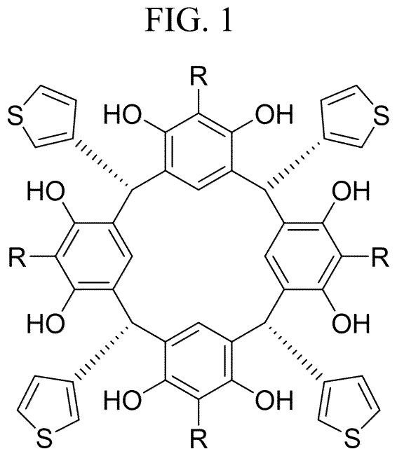

The present invention involves novel calix [4]arenes prepared from the condensation of hydroxyl containing aromatic compounds and heteroaromatic aldehydes. In one embodiment, the present invention is a composition having the formula of FIG. 1 where R is H or an OH group. In another embodiment, the present invention is a tetrameric reaction product of heterocyclic 3-thiophenecarboxaldehyde and an aromatic compound, the reaction product having the formula shown in FIG. 1. In one embodiment, the present invention is a composition having a C-thiopheneresorcin[4]arene. In another embodiment, the present invention is a composition having a C-thiophenepyrogallol[4 ]arene.

Calix[4]arenes are distinct cyclic tetrameric compounds having an alternating structure derived from the reaction of a phenolic compound with an aldehyde. The core structure is typically bowl-shaped tetrameric calix [4]arene formed from aromatic compounds having hydroxy groups and aromatic aldehydes preferably also having heteroatom present. Calix [4]arenes exist in four different conformers: cis-cis-cis, cis-cis-trans, cis-trans-cis and trans-trans-trans. Typically, the thermodynamically most favored conformer is the cis-cis-cis in which all four arene rings are flipped in the same direction such that the calix [4]arene molecule possesses a C₄ symmetry axis, in contrast to the trans-trans-trans isomer which possesses a C₂ symmetry axis. When prepared using hydroxyl aromatic compounds such as resorcinol (1,3-dihydroxy benzene) for example, the hydroxy groups effectively lock the conformation by geometrically-favored hydrogen-bonding interactions between hydroxy groups on adjacent aromatic rings. This locked conformation imparts a “bowl-shaped” structure to the all-cis calix[4]arene. In contrast, when pyrogallol (1,2,3-trihydroxy benzene) was used, the “chair-shaped” conformation was observed wherein two adjacent thiophene moieties are flipping upwards, while their middle pyrogallol ring is tossing downwards and the reverse case is seen in the opposite side of the molecule. The other two bridging pyrogallol units are protruding outwards.

### Applications of Thiophene Functionalized Calixarenes

Calixarenes are a class of macrocyclic compounds that have a basket-like structure with hydrophobic cavities. Thiophene functionalized calixarenes, which incorporate thiophene moieties into their structure, exhibit unique properties that make them suitable for various applications. The thiophene functionalized calixarenes of the present invention can be used in a variety of applications. For example, calixarenes functionalized with thiophene can participate in supramolecular assemblies through non-covalent interactions. These assemblies can be utilized in the construction of advanced materials with unique properties, such as molecular switches or nanoscale devices. These macrocycles can act as hosts for guest molecules through non-covalent interactions. This property can be employed for the design of novel materials, including inclusion complexes with enhanced properties.

Calixarenes functionalized thiophene can be designed to selectively bind specific guest molecules. This molecular recognition property makes them useful in sensing applications, such as detecting certain ions or molecules in solution. Functionalized derivatives can serve as hosts for catalytic species, facilitating various chemical reactions. Their cavity structure can provide a controlled environment for catalytic processes, enhancing reaction selectivity and efficiency.

Calixarenes, including thiophene functionalized variants, can be modified to selectively bind and extract specific metal ions from aqueous solutions. This property is valuable in environmental remediation and metal recovery processes. The hydrophobic cavities within these calixarenes can be utilized to encapsulate and deliver drugs. The controlled release of drugs from these calixarene-based carriers can enhance the therapeutic efficacy and reduce side effects.

Calixarenes functionalized thiophene can be integrated with imaging agents for use in biomedical imaging techniques. Their ability to selectively interact with certain biomolecules makes them potentially valuable in targeted imaging applications. This would depend on the precise structural modifications made to the molecules and the desired properties for a given application. The electrochemical activity of thiophene derivatives in calixarenes can be exploited for the development of sensors. Thiophene functionalized calixarenes may be used as sensing materials for detecting analytes through changes in their electrochemical behavior.

Integrating thiophene moieties into calixarene-based materials can lead to the development of smart materials with responsive properties. These materials may exhibit changes in their structure or function in response to external stimuli, such as light, pH, or temperature.

### Silver Nanoparticles

Silver nanoparticles (AgNPs) consist of pure crystalline silver with distinctive morphologies, having a size ranging from 1 nm to 100 nm. These nanoparticles have received increasing attention in the biomedical field due to their increased and distinctive physicochemical characteristics. These properties include a reduced particle size, elevated surface area, and the manifestation of quantum confinement effects, distinguishing them from bulk or powdered silver materials. This has propelled the popularity of silver nanoparticles for a diverse array of biomedical applications. Silver nanoparticles, in general, exhibit excellent antibacterial, antifungal, and catalytic properties. When combined with thiophene derivatives, additional features can be imparted, expanding their potential applications. For example, thiophene-derived silver nanoparticles can be incorporated into coatings for medical devices, surfaces, or textiles to provide efficient antimicrobial properties. This can be particularly useful in healthcare settings to prevent the spread of infections. Thiophene-derived silver nanoparticles can also be utilized as catalysts in various chemical reactions. Their unique surface properties can enhance catalytic activity, making them valuable in organic synthesis and other chemical processes.

Sensors for detecting specific molecules or analytes can be developed by functionalizing thiophene-derived silver nanoparticles. Their high surface area and conductivity can contribute to the sensitivity and selectivity of the sensor. In addition, thiophene-derived silver nanoparticles may be useful in cancer therapy through photothermal ablation. Their ability to absorb light and convert it into heat can be employed for targeted destruction of cancer cells.

Water purification systems can be developed by using thiophene-derived silver nanoparticles as antimicrobial agents. They can effectively inhibit the growth of bacteria and other microorganisms, improving the quality of drinking water. Also, thiophene-derived silver nanoparticles can be incorporated into drug delivery systems to enhance the stability and controlled release of pharmaceuticals. The nanoparticles can improve the bioavailability and therapeutic efficacy of drugs.

Thiophene-derived silver nanoparticles can be integrated into textiles to impart antimicrobial properties, creating self-cleaning and odor-resistant fabrics. This can be especially beneficial in the production of sportswear and healthcare textiles. Antimicrobial and antifungal food packaging materials can be developed by incorporating thiophene-derived silver nanoparticles. This can extend the shelf life of food products and prevent the growth of spoilage microorganisms. In addition, thiophene-derived silver nanoparticles can be used in energy storage devices, such as batteries and supercapacitors, to improve conductivity and overall performance.

### EXAMPLES

### Example 1—Synthesis of C-thiophenepyrogallol[4]arene

The synthesis of the C-thiophenepyrogallol[4]arene in ethanol was performed through a conventional method (80° C., 24 h) as well as a microwave method (100° C., 1 h). The synthesis of the C-thiopheneresorcin[4]arene in ethanol was also performed through microwave method (100° C., 1 h). In addition, the synthesis of the C-thiophenepyrogallol[4]arene through a microwave method was also carried out in different solvents like acetonitrile (100° C., 1 h) and 2-ethoxyethanol (100° C., 1 h). It was observed that while using acetonitrile as solvent the significant contribution comes from the “chair form” of the product. In contrast, when the same reaction was carried out in 2-ethoxyethanol, both “boat form” as well as “chair form” of the products were observed with almost equal appearances.

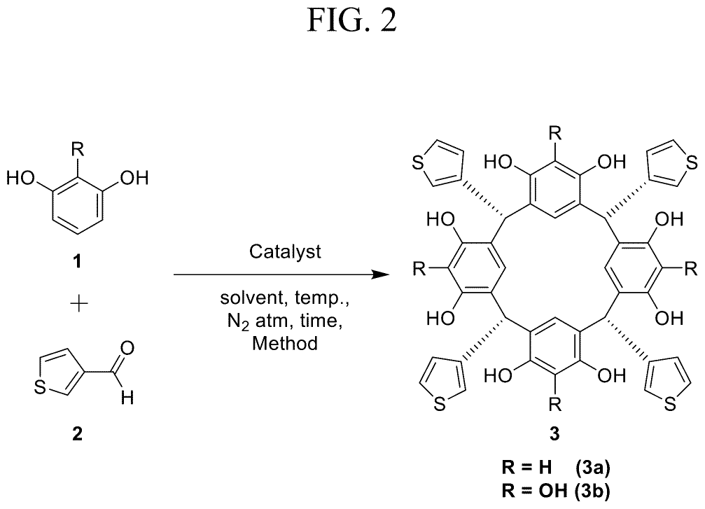

FIG. 2 shows a one-pot synthesis of calix [4]arenes using an acidic catalyst.

TABLE 1
Reaction options for the synthesis

| Entry | Method | Substrate | Catalyst | Solvent | Temperature | Time |
|---|---|---|---|---|---|---|
| 1 | Conventional | Pyrogallol | HCl (Conc.) | Ethanol | 80° C. | 24 h |
| 2 | Microwave | Pyrogallol | HCl (Conc.) | Ethanol | 100° C. | 1 h |
| 3 | Microwave | Resorcinol | HCl (Conc.) | Ethanol | 100° C. | 1 h |
| 4 | Microwave | Pyrogallol | HCl (Conc.) | Acetonitrile | 100° C. | 1 h |
| 5 | Microwave | Pyrogallol | HCl (Conc.) | 2-ethoxyethanol | 100° C. | 1 h |

### Characterization of C-thiopheneresorcin[4]arene and C-thiophenepyrogallol[4]arene:

The final products were characterized by ¹HNMR, MALDI-TOF MS analysis and X-Ray Crystallography.

### C-thiopheneresorcin[4]arene

¹H NMR (CDCl₃, 400 MHZ): δ 5.35 (s, 2H), 5.68 (s, 4H), 5.95 (s, 2H), 6.240-6.247 (m, 4H), 6.35 (d, J=4.92 Hz, 4H), 7.14-7.16 (m, 4H) ppm. MADLI-TOF MS: m/z Expected [C₄₄H₃₂O₈S₄Na⁺]: 839.087; Observed 839.156.

### C-thiophenepyrogallol[4]arene

¹H NMR (CDCl₃, 400 MHZ): δ 5.37 (s, 2H) (chair), 5.69 (s, 4H) (chair), 5.82 (s, 4H) (boat), 5.95 (s, 2H) (chair), 6.04 (s, 4H) (boat), 6.240-6.247 (m, 4H) (chair), 6.36-6.38 (m, 8H) (chair/boat), 6.47 (d, J=4.8 Hz, 4H) (boat), 7.02-7.04 (m, 4H) (chair), 7.14-7.16 (m, 4H) (boat) ppm.

MADLI-TOF MS: m/z Expected [C₄₄H₃₂O₁₂S₄Na⁺]: 903.067; Observed 903.133.

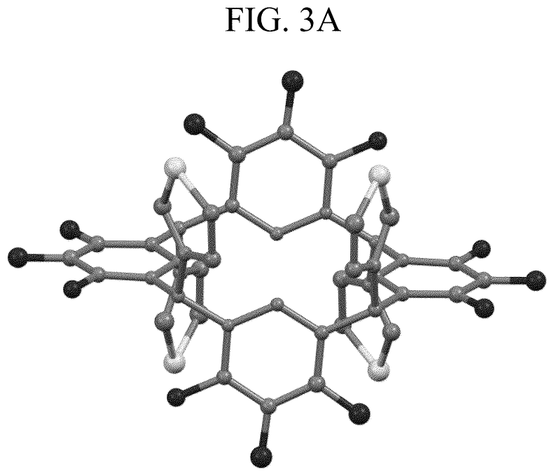

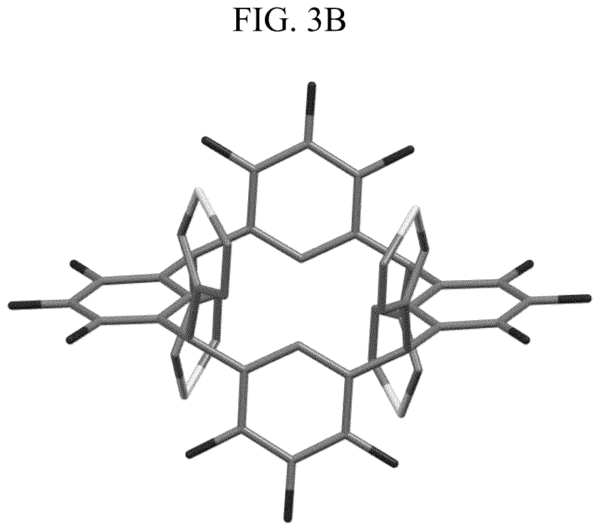

The structure of C-thiophenepyrogallol[4]arene determined by SC-XRD is shown in FIG. 3A, while FIG. 3B shows the same structure with the solvent molecules and hydrogens omitted for clarity.

### Example 2—Synthesis and Characterization of Silver Nanoparticles (Pg₄THP₄—AgNPs)

Thiophene-based pyrogallol[4]arene-capped silver nanoparticles (Pg₄THP₄—AgNPs) were successfully synthesized under white light illumination, thereby exhibiting high sensitivity to various experimental parameters. The key factors that were influencing the synthesis include the silver nitrate (AgNO₃) to C-thiophenepyrogallol[4]arene (Pg₄THP₄) ratio, the ratio of dimethyl sulfoxide (DMSO) to water, and the duration of exposure to the light. Notably, a low concentration of AgNO₃ or a higher concentration of DMSO resulted in the aggregation of Pg₄THP₄—AgNPs. Prolonged exposure to light, approximately 35 minutes beyond the optimum, led to the formation of a dark brown precipitate.

Systematic experimentation was conducted by manipulating different parameters to identify the optimal conditions. The findings highlight that careful control of the AgNO₃ to Pg₄THP₄ ratio, DMSO to water ratio, and exposure time to light is crucial for achieving stable and well-dispersed Pg₄THP₄—AgNPs. The results suggest that thiophene (THP) plays a pivotal role in both the formation and stabilization of nanoparticles, influencing their size and overall characteristics. These optimized parameters provide insights for reproducible and efficient synthesis of Pg₄THP₄—AgNPs, emphasizing the significance of thiophene in this reaction.

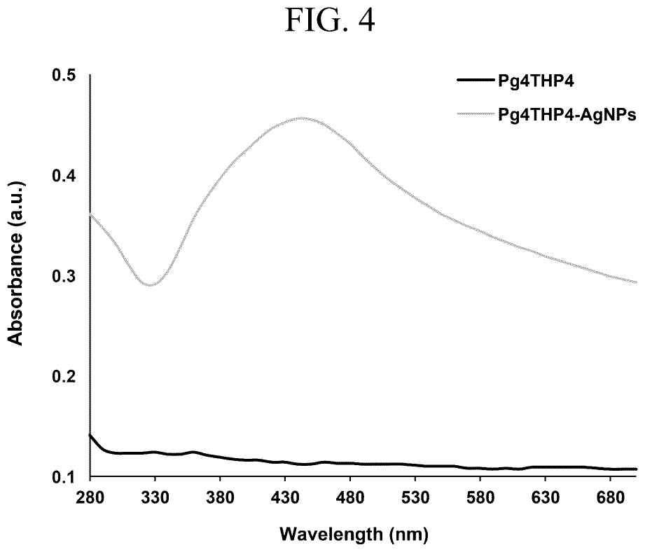

The functionalized nanoparticles, Pg₄THP₄—AgNPs, were characterized based on UV-vis, powder X-ray diffraction (PXRD), and transmission electron microscopy (TEM) studies. The UV-vis spectra of Pg₄THP₄—AgNPs, shown in FIG. 4, exhibit a strong band at 440 nm, which is the characteristic surface plasmon resonance (SPR) absorption band observed for AgNPs.

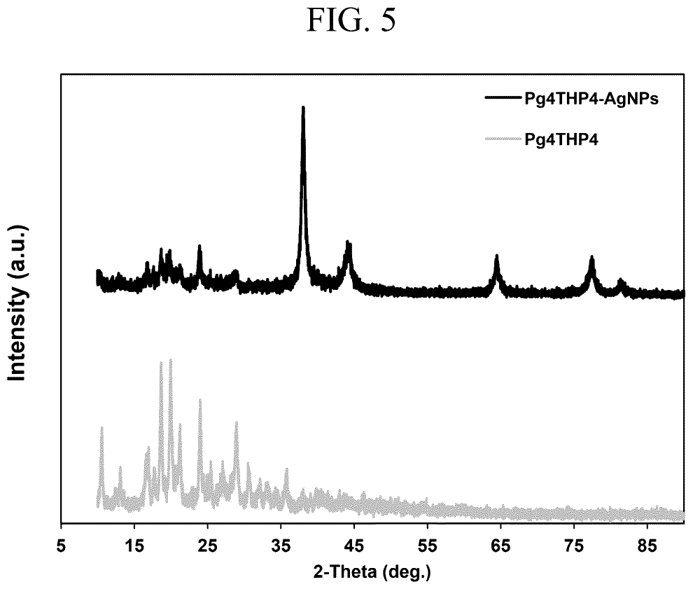

Referring to FIG. 5, a powder XRD pattern of Pg₄THP₄—AgNPs was recorded by collecting the nanoparticle by filtration technique. The formation of silver nanoparticles was revealed by showing the characteristic peaks at 2 θ values of 38.10, 44.07, 64.49, and 77.42° corresponding to the 111, 200, 220, and 311 planes.

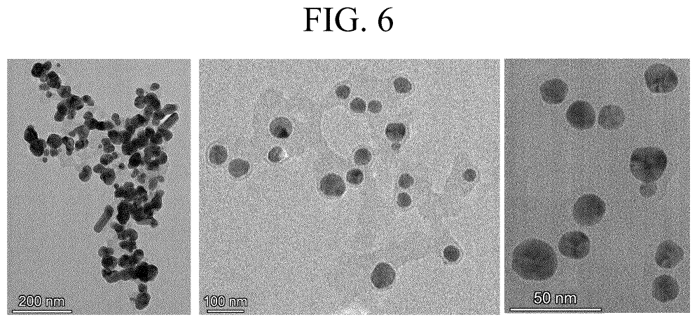

The TEM image of Pg₄THP₄—AgNPs was recorded and is displayed in FIG. 6, which reveals that the nanoparticles are discrete, not aggregated, and almost spherical in shape with average size of 7-23 nm (FIG. 6).

In summary, a simple route for the synthesis of thiophene-derived pyrogallol[4]arene-capped silver nanoparticles (Pg₄THP₄—AgNPs) with the aid of white light is exploited. The thiophene-derived pyrogallol[4]arene (Pg₄THP₄) was attached onto the surface of the AgNPs through the sulfur atoms of the thiophene moiety.

### Example 3—Synthesis of Silver Nanoparticles (Pg₄THP₄—AgNPs)

Silver nanoparticles were synthesized using Pg₄THP₄ as stabilizing agent. In a typical procedure, Pg₄THP₄ (5.3 mg), dissolved in 1.0 mL of dimethyl sulfoxide, was added into the aqueous (99.0 mL) silver nitrate (15.2 mg) solution with a AgNO₃/Pg₄THP₄ molar ratio of 1:15, and the reaction mixture was stirred in 1% (v/v) dimethyl sulfoxide/water for 2 min to mix it well. The reaction mixture was then exposed to white light for 35 min, during which the colorless solution became yellow. The solution was then kept at room temperature for half an hour and then stored at 4° C. for further use.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A composition having the formula: 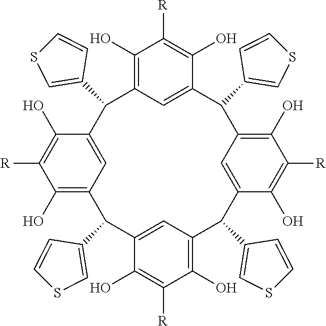
wherein R is H or an OH group.

2. A tetrameric reaction product of heterocyclic 3-thiophenecarboxaldehyde and an aromatic compound, the reaction product being a calix[4]arene.

3. A thiophene functionalized calixarene selected from the group consisting of C-thiopheneresorcin[4]arene and C-thiophenepyrogallol[4]arene.

4. The composition of claim 3, wherein the thiophene functionalized calixarene is C-thiopheneresorcin[4]arene.

5. The composition of claim 3, wherein the thiophene functionalized calixarene is C-thiophenepyrogallol[4]arene.

6. The composition of claim 3 wherein the thiophene functionalized calixarene further comprises silver nanoparticles.

7. A composition comprising thiophene-based pyrogallol[4]arene-capped silver nanoparticles having the formula Pg₄THP₄—AgNP.

8. A pharmaceutical drug comprising the composition of claim 1.

9. An imaging agent comprising the composition of claim 1.

10. An antibiotic coating comprising the composition of claim 7.
